(19) United States
(12) Patent Application Publication
(10) Pub. No.: US 2025/0302406 A1
(43) Pub. Date: Oct. 2, 2025

(54) METHODS AND SYSTEMS FOR TUNGSTEN-BASED RADIATION SHIELD FOR A PRE-PATIENT COLLIMATOR OF AN IMAGING DEVICE

(71) Applicant: GE Precision Healthcare LLC, Waukesha, WI (US)

(72) Inventors: Vishwanath Nayak K, Bangalore (IN); Saiesh Raiker, Bangalore (IN); Chad Smith, Franklin, WI (US)

(21) Appl. No.: 18/920,290

(22) Filed: Oct. 18, 2024

(30) Foreign Application Priority Data
Mar. 29, 2024 (IN) 202441025765

(51) Int. Cl. A61B 6/10; G21F 1/08

(52) U.S. Cl. CPC A61B 6/107; G21F 1/085

(57) ABSTRACT

Various methods and systems are provided for a radiation shielding element having a single continuous shape formed of a first wall and a second wall positioned at a non-zero angle relative to the first wall, the second wall continuous with the first wall along a first axis, and both of the first wall and the second wall comprised of a tungsten polymer blend, wherein tungsten is in an amount of 20% to 60% by volume with respect to polymer.

### CROSS-REFERENCE TO RELATED APPLICATIONS

This application claims priority to Indian application Ser. No. 202441025765, filed on Mar. 29, 2024, the disclosure of which is incorporated herein by reference in its entirety.

### FIELD

Embodiments of the subject matter disclosed herein relate to a radiation shielding element and, in particular, to methods of manufacturing a tungsten-based radiation shielding element via additive fabrication.

### BACKGROUND

Various types of electromagnetic radiation shielding have been developed for use in imaging applications and similar applications, such as x-ray fluorescence (XRF) devices, that also use electromagnetic radiation shielding to reduce undesired spread of radiation in an environment. Traditionally, such forms of shielding include the usage of lead. Lead is a highly effective shielding material due to its high density (e.g., relatively high atomic mass and small atomic radius), which absorbs and scatters various forms of electromagnetic radiation, including x-rays. With recent developments in the field of digital radiography, digital sensors used to replace traditional photographic film are capable of producing x-ray images from a lower level of radiation emission. However, lead lacks structural strength and rigidity such that the shape of a lead-based shield may warp or shift over time, leading to a degradation of a shielding ability of the shield.

Tungsten is another material which may be used to form radiation shielding elements. Tungsten may be combined with a polymer to form planar shielding elements, such as flat sheets which are formed using injection molding and/or extrusion of a tungsten and polymer blend to form radiation shielding elements. Conventional methods for forming radiation shielding elements which have geometries more complex than planar sheets, such as a casing, framing, or other geometries with a 3D shape, may include printing, stamping, or otherwise forming pieces of a radiation shielding element and performing post processing steps such as gluing, welding, or otherwise fastening the pieces together to form the radiation shielding element. Post processing may additionally include cutting, stamp pressing, or otherwise removing material from the radiation shielding element to form through holes. However, this may result in sections of the radiation shielding element through which radiation may leak. Additionally, this process may be undesirably time and resource consuming.

### BRIEF DESCRIPTION

Described herein is a radiation shielding element having a single continuous shape formed of a first wall and a second wall positioned at a non-zero angle relative to the first wall, the second wall continuous with the first wall along a first axis, and both of the first wall and the second wall comprised of a blend of tungsten and a polymer, wherein tungsten is in an amount of 20% to 60% by volume with respect to the polymer. The radiation shielding element includes at least one of a through hole, a groove, an undercut, or a flap. The polymer is one of polyamide (PA) 11, PA12, thermoplastic such as thermoplastic polyurethane (TPU), acrylonitrile butadiene styrene (ABS), or an equivalent plastic.

The radiation shielding element may be formed via additive fabrication. A method for manufacturing a radiation shielding element comprises receiving data defining one or more parameters of a radiation shielding element, generating a digital model of the radiation shielding element from the one or more parameters, the digital model comprising a first wall and a second wall positioned at a non-zero angle relative to the first wall, the second wall continuous with the first wall along a first axis, defining an additive fabrication tool pathway for forming the radiation shielding element as a single, continuous shape according to the digital model, providing to an additive fabrication tool a molten compound comprising a substantially pure volume of tungsten substantially evenly distributed throughout a polymeric mixture, wherein the tungsten is in an amount of 20% to 60% by volume with respect to the polymeric mixture, and actuating the additive fabrication tool to dispense the molten compound into a predetermined location according to the additive fabrication tool pathway, wherein the predetermined location defines a single, continuous geometry of the radiation shielding element having the first wall and the second wall positioned at the non-zero angle relative to the first wall, the second wall continuous with the first wall along the first axis.

The formulation of the tungsten polymer blend (e.g., tungsten is 20% to 60% by volume with respect to the polymer) enables the material used to form the radiation shielding element to be selectively malleable. The tungsten polymer blend, formed as pellets, a filament, a powder, and so on, may be heated to a molten state to increase a malleability of the tungsten polymer blend and enable forming of complex geometries by dispensing the tungsten polymer blend in the molten state from an additive fabrication tool. The formulation may further enable the tungsten polymer blend to rapidly (e.g., within 5 seconds) cool to a solid, non-molten state upon dispense by the additive fabrication tool, where the tungsten polymer blend in the solid state has a rigidity that enables building and retention of the geometry of the radiation shielding element. The tungsten polymer blend has a density between 4000 kg/m³ and at least 12000 kg/m³, and walls of the radiation shielding element have a thickness which enables shielding of radiation which is comparable to that provided by radiation shielding element formed of lead or pure tungsten, for example, while having a lower cost, density, and enables more complex shapes. For example, the thickness may be from 0.2 mm or 0.3 mm with a maximum thickness of 5 mm. In some embodiments, the thickness may be 6 mm or greater. Further, due to the density of the tungsten polymer blend, the radiation shielding element may be lighter than radiation shielding elements formed of other materials, such as blends of tungsten and polymers having a different percentage tungsten by volume, or formed of lead, which may simplify an imaging procedure in which one or more radiation shielding elements are added to/removed from an imaging device.

This summary introduces concepts that are described in more detail in the detailed description. It should not be used to identify essential features of the claimed subject matter, nor to limit the scope of the claimed subject matter. It should be understood that the brief description above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

The present disclosure will be better understood from reading the following description of non-limiting embodiments, with reference to the attached drawings, wherein below:

FIG. 1 shows a pictorial view of an imaging system, according to an embodiment;

FIG. 2 shows a block schematic diagram of an exemplary imaging system, according to an embodiment;

FIG. 3 shows a perspective view of a first example radiation shielding element;

FIG. 4 shows a side view of the first example radiation shielding element;

FIG. 5 shows a graph for an attenuation coefficient of a conventional radiation shielding material;

FIG. 6 shows a graph for an attenuation coefficient of a tungsten and polymer blend, where the tungsten is in an amount of 20% to 60% by volume with respect to the polymeric mixture;

FIG. 7 shows a second example radiation shielding element;

FIG. 8 shows a first perspective view of a third example radiation shielding element;

FIG. 9 shows a second perspective view of the third example radiation shielding element; and

FIG. 10 illustrates a method for forming a radiation shielding element via additive fabrication.

### DETAILED DESCRIPTION

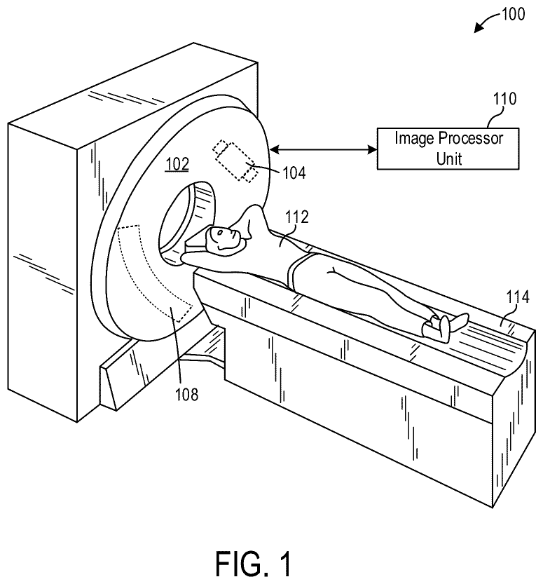

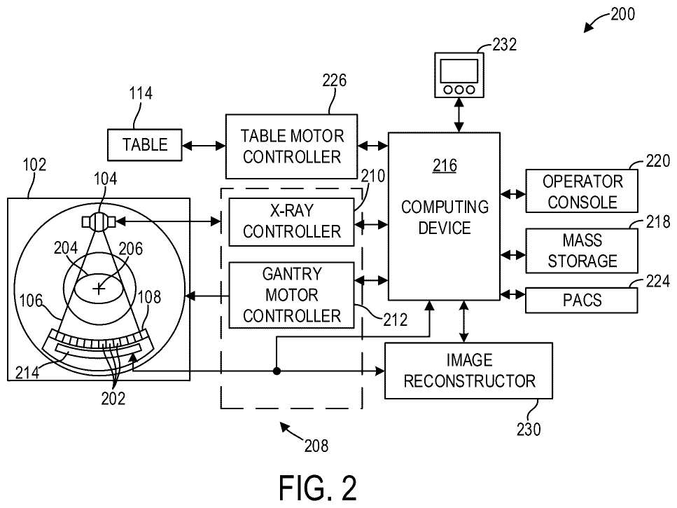

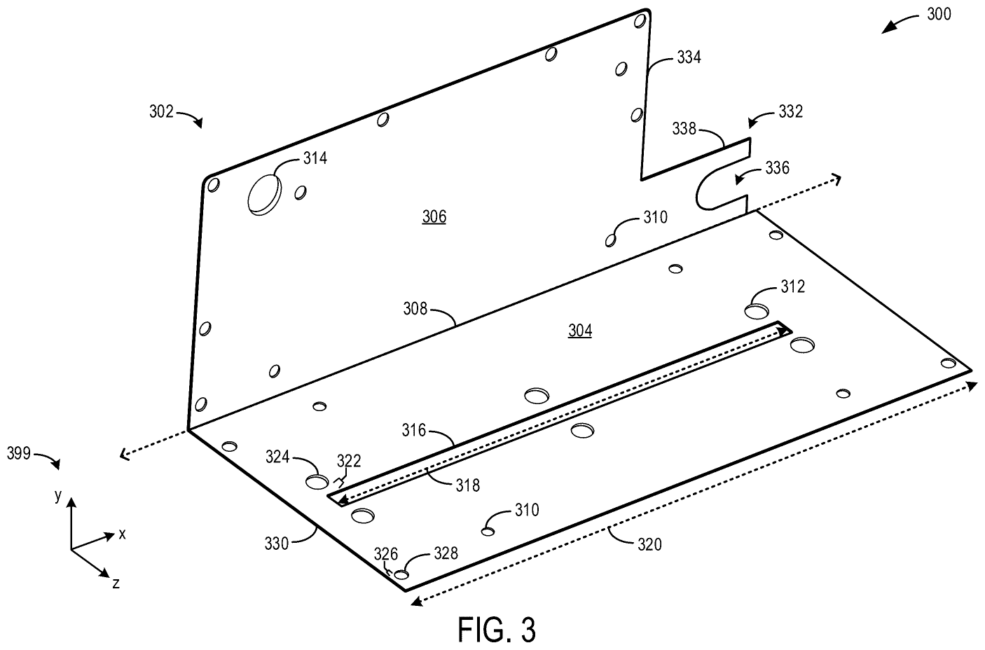

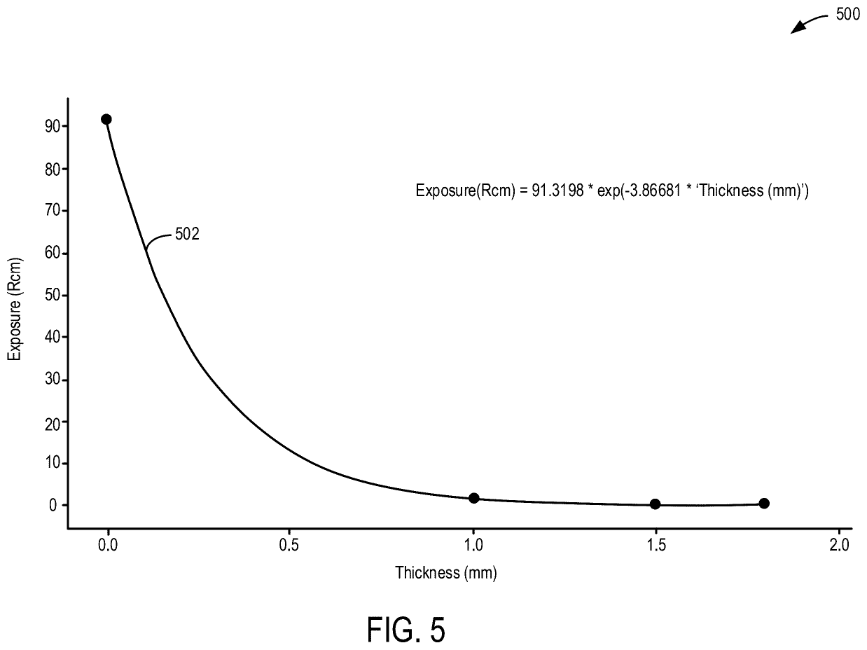

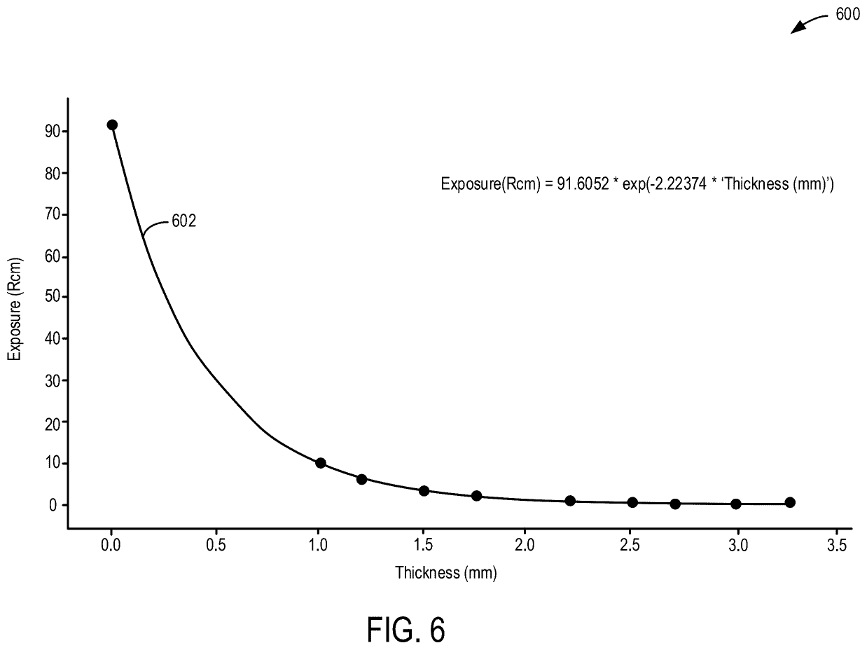

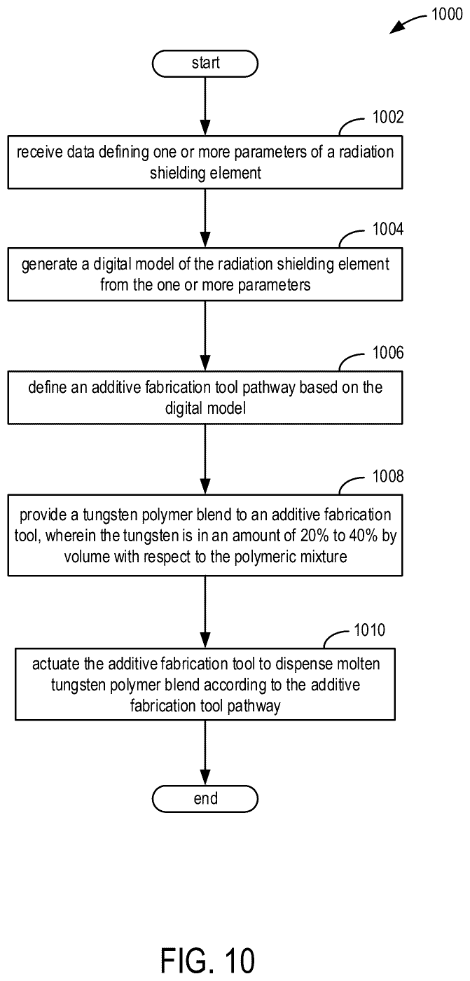

Embodiments of the present disclosure will now be described, by way of example, with reference to the FIGS. 1-10, which relate to various embodiments for a radiation shielding element having a single continuous shape formed of a first wall and a second wall positioned at a non-zero angle relative to the first wall, where the second wall is continuous with the first wall along a first axis, and both of the first wall and the second wall comprised of a blend of tungsten and a polymer, wherein tungsten is in an amount of 20% to 60% by volume with respect to the polymer. FIGS. 1 and 2 illustrate an exemplary imaging system which emits radiation and may have a radiation shielding element, as described herein, coupled thereto. FIGS. 3-4, 7, and 8-9 show example radiation shielding elements, formed of the tungsten polymer blend of 20% to 60% tungsten by volume with respect to the polymer, and each having a single, continuous shape. FIGS. 5 and 6 each include graphs illustrating a radiation attenuation coefficient for coupons of different thicknesses formed of conventional lead or tungsten-based material, or the tungsten polymer blend described herein, respectively. FIG. 10 illustrates a flow chart for a method for forming a radiation shielding element, such as the radiation shielding elements described herein, via additive fabrication.

FIG. 1 illustrates an exemplary computed tomography (CT) system 100 configured for CT imaging. Particularly, the CT system 100 is configured to image a subject 112 such as a patient, an inanimate object, one or more manufactured parts, and/or foreign objects such as dental implants, stents, and/or contrast agents present within the body. In one embodiment, the CT system 100 includes a gantry 102, which in turn, may further include at least one x-ray source 104 configured to project a beam of x-ray radiation 106 (see FIG. 2) for use in imaging the subject 112 laying on a table 114. Specifically, the x-ray source 104 is configured to project the x-ray radiation beams 106 towards a detector array 108 positioned on the opposite side of the gantry 102. Although FIG. 1 depicts only a single x-ray source 104, in certain embodiments, multiple x-ray sources and detectors may be employed to project a plurality of x-ray radiation beams 106 for acquiring projection data at different energy levels corresponding to the patient. In some embodiments, the x-ray source 104 may enable dual-energy gemstone spectral imaging (GSI) by rapid peak kilovoltage (kVp) switching. In some embodiments, the x-ray detector employed is a photon-counting detector which is capable of differentiating x-ray photons of different energies. In other embodiments, two sets of x-ray sources and detectors are used to generate dual-energy projections, with one set at low-kVp and the other at high-kVp.

In certain embodiments, the CT system 100 further includes an image processor unit 110 configured to reconstruct images of a target volume of the subject 112 using an iterative or analytic image reconstruction method. For example, the image processor unit 110 may use an analytic image reconstruction approach such as filtered back projection (FBP) to reconstruct images of a target volume of the patient. As another example, the image processor unit 110 may use an iterative image reconstruction approach such as advanced statistical iterative reconstruction (ASIR), conjugate gradient (CG), maximum likelihood expectation maximization (MLEM), model-based iterative reconstruction (MBIR), and so on to reconstruct images of a target volume of the subject 112. As described further herein, in some examples the image processor unit 110 may use both an analytic image reconstruction approach such as FBP in addition to an iterative image reconstruction approach.

In some CT imaging system configurations, an x-ray source projects a cone-shaped x-ray radiation beam which is collimated to lie within an X-Y-Z plane of a Cartesian coordinate system and generally referred to as an “imaging plane.” The x-ray radiation beam passes through an object being imaged, such as the patient or subject. The x-ray radiation beam, after being attenuated by the object, impinges upon an array of detector elements. The intensity of the attenuated x-ray radiation beam received at the detector array is dependent upon the attenuation of a radiation beam by the object. Each detector element of the array produces a separate electrical signal that is a measurement of the x-ray beam attenuation at the detector location. The attenuation measurements from all the detector elements are acquired separately to produce a transmission profile.

In some CT systems, the x-ray source and the detector array are rotated with a gantry within the imaging plane and around the object to be imaged such that an angle at which the radiation beam intersects the object constantly changes. A group of x-ray radiation attenuation measurements (e.g., projection data) from the detector array at one gantry angle is referred to as a “view.” A “scan” of the object includes a set of views made at different gantry angles, or view angles, during one revolution of the x-ray source and detector. It is contemplated that the benefits of the methods described herein accrue to medical imaging modalities other than CT, so as used herein the term “view” is not limited to the use as described above with respect to projection data from one gantry angle. The term “view” is used to mean one data acquisition whenever there are multiple data acquisitions from different angles, whether from a CT, positron emission tomography (PET), or single-photon emission CT (SPECT) acquisition, and/or any other modality including modalities yet to be developed as well as combinations thereof in fused embodiments.

The projection data is processed to reconstruct an image that corresponds to a two-dimensional slice taken through the object or, in some examples where the projection data includes multiple views or scans, a three-dimensional rendering of the object. One method for reconstructing an image from a set of projection data is referred to in the art as the filtered back projection technique. Transmission and emission tomography reconstruction techniques also include statistical iterative methods such as maximum likelihood expectation maximization (MLEM) and ordered-subsets expectation-reconstruction techniques as well as iterative reconstruction techniques. This process converts the attenuation measurements from a scan into integers called “CT numbers” or “Hounsfield units,” which are used to control the brightness of a corresponding pixel on a display device.

To reduce the total scan time, a “helical” scan may be performed. To perform a “helical” scan, the patient is moved while the data for the prescribed number of slices is acquired. Such a system generates a single helix from a cone beam helical scan. The helix mapped out by the cone beam yields projection data from which images in each prescribed slice may be reconstructed.

As used herein, the phrase “reconstructing an image” is not intended to exclude embodiments in which data representing an image is generated but a viewable image is not. Therefore, as used herein, the term “image” broadly refers to both viewable images and data representing a viewable image. However, many embodiments generate (or are configured to generate) at least one viewable image.

FIG. 2 illustrates an exemplary imaging system 200 similar to the CT system 100 of FIG. 1. In accordance with aspects of the present disclosure, the imaging system 200 is configured for imaging a subject 204 (e.g., the subject 112 of FIG. 1). In one embodiment, the imaging system 200 includes the detector array 108 (see FIG. 1). The detector array 108 further includes a plurality of detector elements 202 that together sense the x-ray radiation beams 106 (see FIG. 2) that pass through the subject 204 (such as a patient) to acquire corresponding projection data. Accordingly, in one embodiment, the detector array 108 is fabricated in a multi-slice configuration including the plurality of rows of cells or detector elements 202. In such a configuration, one or more additional rows of the detector elements 202 are arranged in a parallel configuration for acquiring the projection data.

In certain embodiments, the imaging system 200 is configured to traverse different angular positions around the subject 204 for acquiring desired projection data. Accordingly, the gantry 102 and the components mounted thereon may be configured to rotate about a center of rotation 206 for acquiring the projection data, for example, at different energy levels. Alternatively, in embodiments where a projection angle relative to the subject 204 varies as a function of time, the mounted components may be configured to move along a general curve rather than along a segment of a circle.

As the x-ray source 104 and the detector array 108 rotate, the detector array 108 collects data of the attenuated x-ray beams. The data collected by the detector array 108 undergoes pre-processing and calibration to condition the data to represent the line integrals of the attenuation coefficients of the scanned subject 204. The processed data are commonly called projections.

In some examples, the individual detectors or detector elements 202 of the detector array 108 may include photon-counting detectors which register the interactions of individual photons into one or more energy bins. It should be appreciated that the methods described herein may also be implemented with energy-integrating detectors.

The acquired sets of projection data may be used for basis material decomposition (BMD). During BMD, the measured projections are converted to a set of material-density projections. The material-density projections may be reconstructed to form a pair or a set of material-density map or image of each respective basis material, such as bone, soft tissue, and/or contrast agent maps. The density maps or images may be, in turn, associated to form a volume rendering of the basis material, for example, bone, soft tissue, and/or contrast agent, in the imaged volume.

Once reconstructed, the basis material image produced by the imaging system 200 reveals internal features of the subject 204, expressed in the densities of two basis materials. The density image may be displayed to show these features. In traditional approaches to diagnosis of medical conditions, such as disease states, and more generally of medical events, a radiologist or physician would consider a hard copy or display of the density image to discern characteristic features of interest. Such features might include lesions, sizes and shapes of particular anatomies or organs, and other features that would be discernable in the image based upon the skill and knowledge of the individual practitioner.

In one embodiment, the imaging system 200 includes a control mechanism 208 to control movement of the components such as rotation of the gantry 102 and the operation of the x-ray source 104. In certain embodiments, the control mechanism 208 further includes an x-ray controller 210 configured to provide power and timing signals to the x-ray source 104. Additionally, the control mechanism 208 includes a gantry motor controller 212 configured to control a rotational speed and/or position of the gantry 102 based on imaging requirements.

In certain embodiments, the control mechanism 208 further includes a data acquisition system (DAS) 214 configured to sample analog data received from the detector elements 202 and convert the analog data to digital signals for subsequent processing. The DAS 214 may be further configured to selectively aggregate analog data from a subset of the detector elements 202 into so-called macro-detectors, as described further herein. The data sampled and digitized by the DAS 214 is transmitted to a computer or computing device 216. In one example, the computing device 216 stores the data in a storage device 218. The storage device 218, for example, may include a hard disk drive, a floppy disk drive, a compact disk-read/write (CD-R/W) drive, a Digital Versatile Disc (DVD) drive, a flash drive, and/or a solid-state storage drive.

Additionally, the computing device 216 provides commands and parameters to one or more of the DAS 214, the x-ray controller 210, and the gantry motor controller 212 for controlling system operations such as data acquisition and/or processing. In certain embodiments, the computing device 216 controls system operations based on operator input. The computing device 216 receives the operator input, for example, including commands and/or scanning parameters via an operator console 220 operatively coupled to the computing device 216. The operator console 220 may include a keyboard (not shown) or a touchscreen to allow the operator to specify the commands and/or scanning parameters.

Although FIG. 2 illustrates only one operator console 220, more than one operator console may be coupled to the imaging system 200, for example, for inputting or outputting system parameters, requesting examinations, plotting data, and/or viewing images. Further, in certain embodiments, the imaging system 200 may be coupled to multiple displays, printers, workstations, and/or similar devices located either locally or remotely, for example, within an institution or hospital, or in an entirely different location via one or more configurable wired and/or wireless networks such as the Internet and/or virtual private networks, wireless telephone networks, wireless local area networks, wired local area networks, wireless wide area networks, wired wide area networks, and so on.

In one embodiment, for example, the imaging system 200 either includes, or is coupled to, a picture archiving and communications system (PACS) 224. In an exemplary implementation, the PACS 224 is further coupled to a remote system such as a radiology department information system, hospital information system, and/or to an internal or external network (not shown) to allow operators at different locations to supply commands and parameters and/or gain access to the image data.

The computing device 216 uses the operator-supplied and/or system-defined commands and parameters to operate a table motor controller 226, which in turn, may control a table 114 which may be a motorized table. Specifically, the table motor controller 226 may move the table 114 for appropriately positioning the subject 204 in the gantry 102 for acquiring projection data corresponding to the target volume of the subject 204.

As previously noted, the DAS 214 samples and digitizes the projection data acquired by the detector elements 202. Subsequently, an image reconstructor 230 uses the sampled and digitized x-ray data to perform high-speed reconstruction. Although FIG. 2 illustrates the image reconstructor 230 as a separate entity, in certain embodiments, the image reconstructor 230 may form part of the computing device 216. Alternatively, the image reconstructor 230 may be absent from the imaging system 200 and instead the computing device 216 may perform one or more functions of the image reconstructor 230. Moreover, the image reconstructor 230 may be located locally or remotely, and may be operatively connected to the imaging system 200 using a wired or wireless network. Particularly, one exemplary embodiment may use computing resources in a “cloud” network cluster for the image reconstructor 230.

In one embodiment, the image reconstructor 230 stores the images reconstructed in the storage device 218. Alternatively, the image reconstructor 230 may transmit the reconstructed images to the computing device 216 for generating useful patient information for diagnosis and evaluation. In certain embodiments, the computing device 216 may transmit the reconstructed images and/or the patient information to a display or display device 232 communicatively coupled to the computing device 216 and/or the image reconstructor 230. In some embodiments, the reconstructed images may be transmitted from the computing device 216 or the image reconstructor 230 to the storage device 218 for short-term or long-term storage.

A radiation shielding element may be selectively coupled, via one or more screws, bolts, clips, latches, and so on, to an imaging device and/or a user input device thereof. For example, a radiation shielding device may be coupled to the table 114 to shield an area of the subject 112 which is not to be imaged from radiation. Additionally or alternatively, a radiation shielding element may be coupled to the operator console 220. A radiation shielding element may further be coupled to additional elements or areas of an environment in which radiation is used, such as to selectively shield areas of the environment from radiation. As different environments in which radiation is used may include the same or different devices formed by the same or different manufacturers, arranged in different positions around rooms of different shapes and sizes, and where the devices are used at different frequencies by different operators and in different ways, a different size and shape radiation shielding element may be desired in different environments. It is desirable that a radiation shielding element have substantially the same shielding abilities as lead or other blends of tungsten and polymer while having a desired size and shape. Described herein are example radiation shielding elements formed of “the tungsten polymer blend”, wherein tungsten is in an amount of 20% to 60% tungsten by volume with respect to the polymer. The tungsten polymer blend described herein is malleable and may be stored in one of a powder, pellet, spool, and so on. Malleability of the tungsten polymer blend (e.g., heating to a molten state and rapidly cooling to a solid state without use of external tools to cool) and rigidity of the tungsten polymer blend in the solid state enables radiation shielding elements to be formed which have continuous (e.g., seamless) geometry. Additionally, the tungsten polymer blend adheres to the Restriction of Hazardous Substances (RoHS) Directive. The tungsten polymer blend is recyclable and reduces a carbon footprint of manufacturing. The methods described herein for forming the radiation shielding of tungsten polymer blend may have a plurality of advantages compared to radiation shielding elements formed of other materials, such as lead, or formed using methods with pre-and/or post-processing steps. For example, the methods described herein for forming radiation shielding elements may produce radiation shielding elements with continuous, complex geometries may be scaled to desired sizes and shapes. For another example, the methods described herein for forming radiation shielding elements may reduce an amount of hazardous waste generated in the manufacturing process. As described herein, a wall which is continuous with another wall along an axis is to be understood as being seamlessly joined. For example, the first wall and the second wall are formed of the tungsten and polymer blend, and the second wall is formed as a continuation of the first wall. Additionally, the radiation shielding element may include through holes which extend through a thickness of a wall from a first side to a second side of the wall. When forming radiation shielding elements using methods other than additive fabrication, a through hole may be formed during post processing and/or complex stamp pressing.

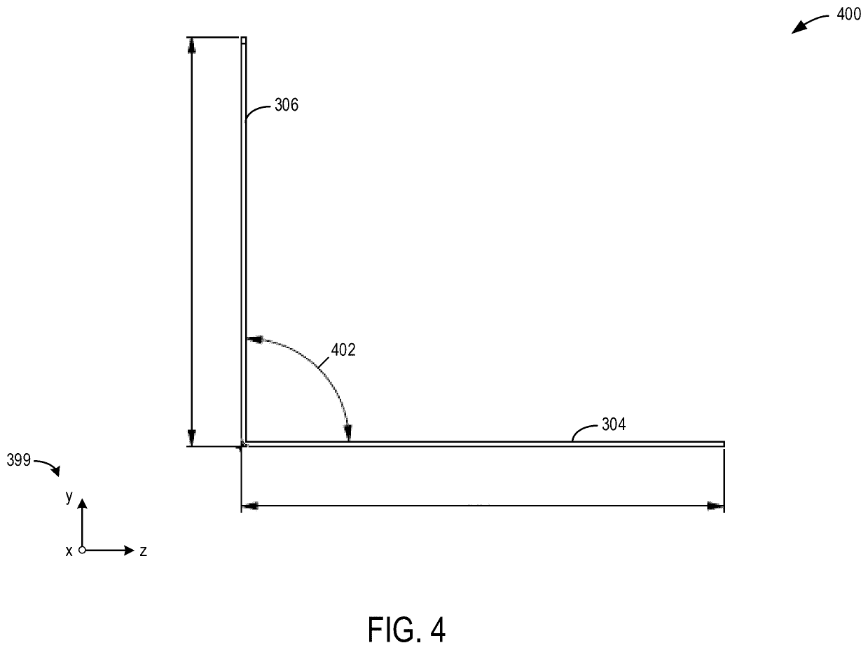

A first example of a radiation shielding element 302 is shown in a first perspective view 300 of FIG. 3. An axis system 399 is provided in FIGS. 3-4 for reference. The y-axis may be a vertical axis (e.g., parallel to a gravitational axis), the x-axis may be a lateral axis (e.g., horizontal axis), and the z-axis may be a longitudinal axis, in one example. However, the axes may have other orientations, in other examples. An axis may be represented as a filled circle when normal to and extending toward a view. Likewise, an axis may be represented by an unfilled circle when normal to and extending away from a view, such as in the second perspective view 400.

The radiation shielding element 302 is formed of a blend of tungsten and a polymer, wherein tungsten is in an amount of 20% to 60% by volume with respect to the polymer. The polymer may be one of polyamide (PA) 11, PA12, thermoplastic such as thermoplastic polyurethane (TPU), acrylonitrile butadiene styrene (ABS), or an equivalent plastic or polyamide. The radiation shielding element 302 has a single continuous shape formed of a first wall 304 and a second wall 306 positioned at a non-zero angle relative to the first wall 304 and continuous with the first wall 304 along a first axis 308. The first wall 304 and the second wall 306 are thus seamlessly joined. Each of the first wall 304 and the second wall 306 are planar, and the radiation shielding element 302 comprises at least one of an undercut, a groove, a through hole, or a flap. The radiation shielding element 302 may be formed by additive fabrication, for example, according to the method 1000 of FIG. 10. Thus, features such as an undercut, a groove, a through hole, and/or a flap may be formed in the radiation shielding element 302 during formation of the first wall 304 and the second wall 306. This eliminates a post-processing step (e.g., after formation of the radiation shielding element 302) such as stamp pressing or tooling to generate these features in a radiation shielding element formed using methods other than additive fabrication.

Features of the radiation shielding element 302 include a plurality of through holes in various sizes and shapes. Through holes may be used to mount the radiation shielding element 302 to an imaging device and/or user interface, such as described with respect to FIGS. 1-2. In some embodiments, cables which connect elements of an imaging device to each other and/or to other medical devices may be threaded through through holes, for example. The radiation shielding element 302 includes circular through holes in a small size 310, a medium size 312, and a large size 314. Other example radiation shielding elements may include circular through holes which are larger than the large size 314, smaller than the small size 310, and any size therebetween. The first wall 304 also includes a rectangular through hole 316. The rectangular through hole 316 extends a through hole length 318. The through hole length 318 is less than a wall length 320 of the first wall 304, and greater than half of the wall length 320. In the example shown in FIG. 3, the first wall 304 may further include a plurality of circular through holes along the through hole length 318 on each side of the rectangular through hole 316. The makeup of the tungsten polymer blend is such that the first wall 304 has a structural rigidity which enables the first wall 304 to have multiple through holes distributed across a surface area of the first wall 304 while maintaining a planar integrity of the first wall 304. For example, as described herein, the first wall 304 has multiple through holes which extend over a large surface area (e.g., the rectangular through hole 316 extending greater than half the wall length 320 of the first wall 304) and supported by a small area of the first wall 304 (e.g., a first width 322 of the first wall 304 between a medium size circular through hole 324 and the rectangular through hole 316, a second width 326 of the first wall 304 between a small size circular through hole 328 and a first edge 330 of the first wall 304), and a rigidity of the first wall 304 provided by the makeup of the tungsten polymer blend may prevent areas of the first wall 304 from sagging, bowing, or otherwise deforming out of plane (e.g., the x-z plane, with respect to the axis system 399).

The second wall 306 of the radiation shielding element 302 also includes a plurality of circular through holes of varying sizes. The second wall 306 has a rectangular shape with an extension 332 from a second edge 334, the extension 332 having a curved notch 336. As described above, the makeup of the tungsten polymer blend enables such geometry to be formed during formation of the second wall 306 and to be structurally stable (e.g., an arm 338 of the extension 332 doesn't droop, fold, or otherwise deform towards the curved notch 336). Geometry enabled by a rigidity of the radiation shielding element 302 provided by the composition of the tungsten polymer blend is further described with respect to FIG. 4.

FIG. 4 shows a second perspective view 400 of the radiation shielding element 302. The rigidity of the tungsten polymer blend enables the second wall 306 to be formed at a non-zero angle 402 with respect to the first wall 304. For example, the radiation shielding element 302 has the second wall 306 positioned at a 90-degree angle with respect to the first wall 304, wherein the non-zero angle 402 is the 90-degree angle. In other embodiments, the non-zero angle 402 may be greater than 0-degrees and less than 90-degrees, or greater than 90-degrees and less than 180-degrees. The composition of the tungsten polymer blend (e.g., the percentage of tungsten by volume with respect to polymer) enables the tungsten polymer blend to cool from a molten state to a solid state rapidly (e.g., within 5 seconds) following dispensing of the tungsten polymer blend from an additive fabrication tool, as further described with respect to FIG. 10. In the solid state, the tungsten polymer blend is rigid enough to support structures positioned at non-zero angles with respect to each other, such as the first wall 304 and the second wall 306 of the radiation shielding element 302.

As further described with respect to FIGS. 5-6, the tungsten polymer blend enables attenuation (e.g., shielding) of radiation for a broader range of thicknesses, compared to conventional radiation shielding elements. A variable material density may be achieved for any desired attenuation, and any desired geometry may be formed via additive fabrication using the tungsten polymer blend, as the tungsten polymer blend is able to transition from a solid state to a liquid state via exposure to heat.

FIG. 5 shows a graph 500 illustrating an attenuation coefficient for lead. A thickness of a coupon of material is shown along the x-axis in millimeters (mm). Data of the graph 500 is collected by exposing a coupon of each thickness (e.g., 1.0 mm, 1.5 mm, 1.75 mm) to an x-ray beam of the same strength, and measuring a radiation exposure on a side of the coupon opposite a source of the x-ray beam (e.g., such that the coupon is between a measuring tool and the x-ray beam source). Exposure is shown on the y-axis in reciprocal centimeters (Rcm). A line 502 of the graph 500 is an exponential function which can be used to solve for the attenuation coefficient. The attenuation coefficient shows how easily a volume (e.g., the coupon) can be penetrated by an x-ray beam.

When no coupon is positioned between the measuring tool and the x-ray beam (e.g., at 0.0 mm), the exposure is approximately 91 Rcm. Using collected exposure data for coupons which are 0.5 mm, 1.0 mm, 1.5 mm, and 2.0 mm thick, an exponential decay function of intensity may be generated, wherein the slope is the attenuation coefficient. In the example of FIG. 5, the attenuation coefficient for conventional lead is approximately 3.9.

Turning to FIG. 6, a graph 600 is shown illustrating an attenuation coefficient for the tungsten polymer blend described herein, where tungsten is 20% to 60% by volume, with respect to the polymer. Like the graph 500 of FIG. 5, a thickness of a coupon of material is shown along the x-axis in mm, exposure is shown on the y-axis in Rcm, and data of the graph 600 is collected by exposing a coupon of each thickness (e.g., 0.5 mm, 3.5 mm, and thicknesses therebetween) to an x-ray beam of the same strength and measuring a radiation exposure on a side of the coupon opposite a source of the x-ray beam.

When no coupon is positioned between the measuring tool and the x-ray beam (e.g., at 0.0 mm), the exposure is approximately 92 Rcm. Using collected exposure data for coupons which are 0.5 mm, 1.0 mm, 1.5 mm, 2.0 mm, 2.5 mm, 3.0 mm, and 3.5 mm thick, an exponential decay function of intensity may be generated, shown by a line 602, wherein the slope is the attenuation coefficient. In the example of FIG. 6, the attenuation coefficient for the tungsten and polymer blend described herein with 20% to 60% tungsten by volume is approximately 2.2.

A large attenuation coefficient represents a beam becoming ‘attenuated’ as it passes through a given medium (e.g., the coupon), while a small value represents that the medium had little effect on shielding. The attenuation coefficient measures the exponential decay of intensity, that is, the value of downward e-folding distance of the original intensity as the energy passes through a unit (e.g. one meter) thickness of material, so that an attenuation coefficient of 1 m⁻¹ means that after passing through one meter, the radiation will be reduced by a factor of e, and for material with a coefficient of 2 m⁻¹, it will be reduced twice by e, or e².

As shown in the graph 500, exposure of 91 Rcm is reduced to 0.17 Rcm using a coupon formed of lead which has a thickness of 1.8 mm and an attenuation coefficient of 3.866. Coupons formed of lead have a half value layer thickness of 0.179 mm (e.g., exposure is halved from 91 Rcm by a 0.179 mm coupon). The graph 600 shows an x-ray attenuation capability of the tungsten polymer blend described herein, with a half value layer thickness of 0.311 mm (e.g., exposure is halved from 92 Rcm by a 0.311 mm thick coupon). Exposure of 92 Rcm is reduced to 0.186 Rcm using a coupon formed of the tungsten polymer blend which has a thickness of 3.25 mm and an attenuation coefficient of 2.223. An embodiment of the tungsten polymer blend used to capture the data of the graph 600 may have a low amount of tungsten by volume with respect to the polymer, for example 20%. At this amount of tungsten by volume, the attenuation capability of the tungsten polymer blend is within two units of the attenuation coefficient of lead. The attenuation capability of the tungsten polymer blend may be further increased (e.g., attenuate/shield an increased amount of radiation at a given thickness) by increasing the amount of tungsten by volume with respect to the polymer. Thus, the attenuation coefficient of the tungsten polymer blend may be made similar to, or greater than, that of conventional lead by adjusting the percentage of tungsten.

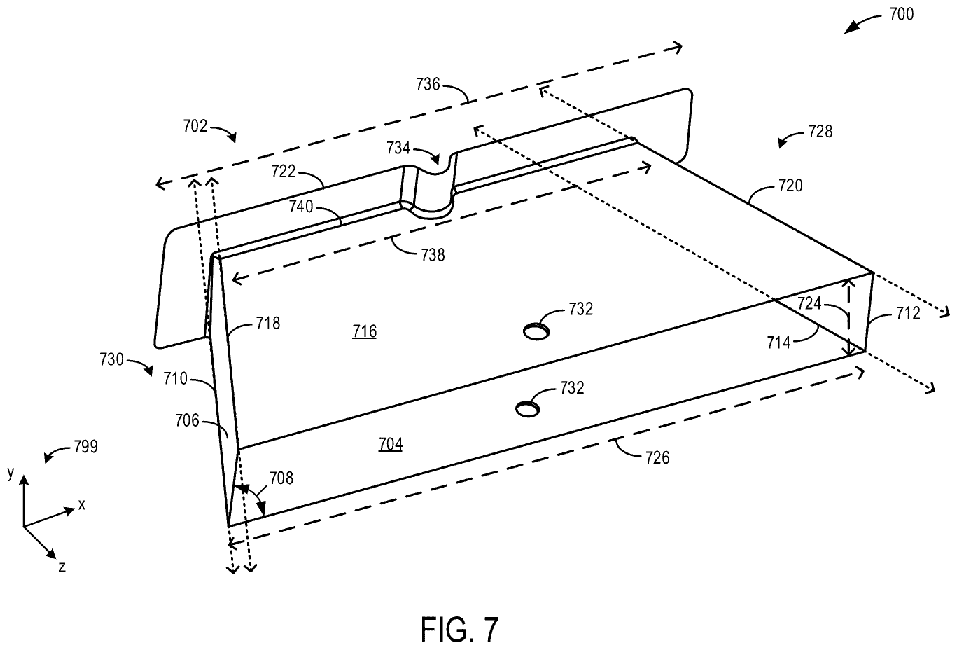

Turning to FIG. 7, a perspective view 700 is shown of a second example of a radiation shielding element 702. An axis system 799 is provided in FIG. 7 for reference. The y-axis may be a vertical axis (e.g., parallel to a gravitational axis), the x-axis may be a lateral axis (e.g., horizontal axis), and the z-axis may be a longitudinal axis, in one example. However, the axes may have other orientations, in other examples. The radiation shielding element 702 has a single continuous shape formed of a first wall 704 and a second wall 706 positioned at a non-zero angle 708 relative to the first wall 704 and continuous with the first wall 704 along a first axis 710. Additionally, the radiation shielding element 702 includes a third wall 712 continuous with the first wall 704 along a second axis 714 of the first wall 704 different from the first axis 710, a fourth wall 716 continuous with the second wall 706 along a third axis 718 (e.g., of the second wall 706, opposite from the first axis 710) and continuous with the third wall 712 along a fourth axis 720 (e.g., of the third wall 712, opposite from the second axis 714). The radiation shielding element 702 further includes a fifth wall 722 continuous with each of the first wall 704, the second wall 706, the third wall 712, and the fourth wall 716. Each of the first wall 704, the second wall 706, the third wall 712 and the fourth wall 716 are planar, where the second wall 706 and the third wall 712 are rectangular and the first wall 704 and the fourth wall 716 are trapezoidal. For example, with respect to the axis system 799, the first wall 704 and the fourth wall 716 are planar in the x-z plane and positioned parallel. The fifth wall 722 is rectangular and has a curved extension 734 which extends out of plane (e.g., the x-y plane, with respect to the axis system 799), as further described herein.

The radiation shielding element 702 is formed of the tungsten polymer blend, wherein tungsten is in an amount of 20% to 60% by volume with respect to the polymer. As described above, the polymer may be one of PA11, PA12, TPU, ABS, or an equivalent plastic. The rigidity of the tungsten polymer blend when in a solid form enables the second wall 706 to be formed at the non-zero angle 708 with respect to the first wall 704. For example, the radiation shielding element 702 has the second wall 706 positioned at a 90-degree angle with respect to the first wall 704. In other embodiments, the non-zero angle 708 may be greater than 0 degrees and less than 90 degrees, or greater than 90 degrees and less than 180 degrees. The composition of the tungsten polymer blend (e.g., the percentage of tungsten by volume with respect to polymer) enables the tungsten polymer blend to cool from a molten state to a solid state rapidly (e.g., within 5 seconds) following dispensing of the tungsten polymer blend in the molten state from an additive fabrication tool, as further described with respect to FIG. 10. In the solid state, the tungsten polymer blend is rigid enough to support structures positioned at non-zero angles with respect to each other, such as the first wall 704 and the second wall 706 of the radiation shielding element 702.

The rigidity of the radiation shielding element 702 as provided by the tungsten polymer blend in the solid state (e.g., not having heat applied thereto) further enables formation of the third wall 712 and the fourth wall 716. The fourth wall 716 is spaced apart from the first wall 704 by a height 724 of the second wall 706 and the third wall 712. The second wall 706 and the third wall 712 are spaced apart by a length 726 of the first wall 704 and the fourth wall 716. The second wall 706 and the third wall 712 extend between a non-parallel pair of sides of the trapezoid (e.g., between a first side 728 of each of the first wall 704 and the fourth wall 716, and between a second side 730 of each of the first wall 704 and the fourth wall 716). The third wall 712 is formed at a non-zero angle with respect to the first wall 704 and the fourth wall 716, and the fourth wall 716 is formed parallel to the first wall 704, and at a non-zero angle with respect to the second wall 706. In the example of FIG. 7, each of the non-zero angles between walls are equal to 90-degrees.

The first wall 704 and fourth wall 716 may be spaced apart as shown in FIG. 7 without bowing, sagging, or otherwise deforming out of plane (e.g., the x-z plane, with respect to the axis system 799) without support therebetween from support elements other than the second wall 706 and the third wall 712 due to a structural integrity of the first wall 704 and the second wall 706 provided by a rigidity of the tungsten and polymer blend when in the solid state. Additionally, each of the first wall 704 and the fourth wall 716 include a circular through hole 732. In the example of FIG. 7, the circular through hole 732 of each of the first wall 704 and the fourth wall 716 are aligned along the y-axis. In some examples, a wire or other connector of an imaging device (e.g., the CT system 100) may pass through the circular through holes 732. In further examples, a screw or other coupling device may pass through one or both of the circular through holes 732 to couple the radiation shielding element 702 to an imaging device or other radiation emitting source.

A rectangular shape of the fifth wall 722 is planar with respect to the x-y plane of the axis system 799, and a curved extension 734 of the fifth wall 722 extends out of the x-y plane in a direction of the z-axis. The fifth wall 722, including the curved extension 734, is continuous with each of the first wall 704, the second wall 706, the third wall 712, and the fourth wall 716. The fifth wall 722 further extends a wall length 736, which is longer than an edge length 738 of the first wall 704 and the fourth wall 716 along a top edge 740. The curved extension 734 is positioned at an approximate center of the wall length 736, and extends in the direction of the z-axis into the fourth wall 716. The curved extension 734 is continuous with both of the fourth wall 716 and the fifth wall 722, and thus the radiation shielding element 702 has no seams or gaps between the fifth wall 722 and the fourth wall 716. As further described with respect to the method of FIG. 10, the radiation shielding element 702 may be formed via additive fabrication in which pellets, powder, spooled filament, or other solid state blend of tungsten and polymer is heated to become molten, and molten blend is extruded from an additive fabrication tool into a predetermined geometry, the molten blend rapidly (e.g., within 5 seconds of being extruded) cooling to solid state. Formation of the curved extension 734 is included in formation of the fifth wall 722 and formation of the fourth wall 716 (and optionally, in formation of the first wall 704, in embodiments where the curved extension 734 extends into the first wall 704). Conventional methods for forming a radiation shielding element may include forming the fourth wall 716 as a trapezoid, forming the fifth wall 722 as one or more rectangular pieces, forming a curved extension piece (e.g., a geometry of the curved extension 734 as an independent piece, separate from the fifth wall 722), then cutting out a shape of the curved extension in the fourth wall 716. The fourth wall 716, the fifth wall 722, and the curved extension piece may be coupled by gluing, welding, or other coupling method. Using additive fabrication and the tungsten polymer blend described herein, the radiation shielding element may be formed as a single, continuous, seamless piece with geometry including linear and curved elements, as well as linear and/or curved through holes in planar or curved elements. Similar to the circular through holes 732 of the first wall 704 and the fourth wall 716, the curved extension 734 of the fifth wall 722 may act as a channel through which to pass one or more wires or other coupling elements of a user input device and/or imaging device. For example, the radiation shielding element 702 may be positioned such that the rectangular shape of the fifth wall 722 is positioned in face-sharing contact with a user input device, and the curved extension 734 provides a gap between the user input device and the radiation shielding element 702.

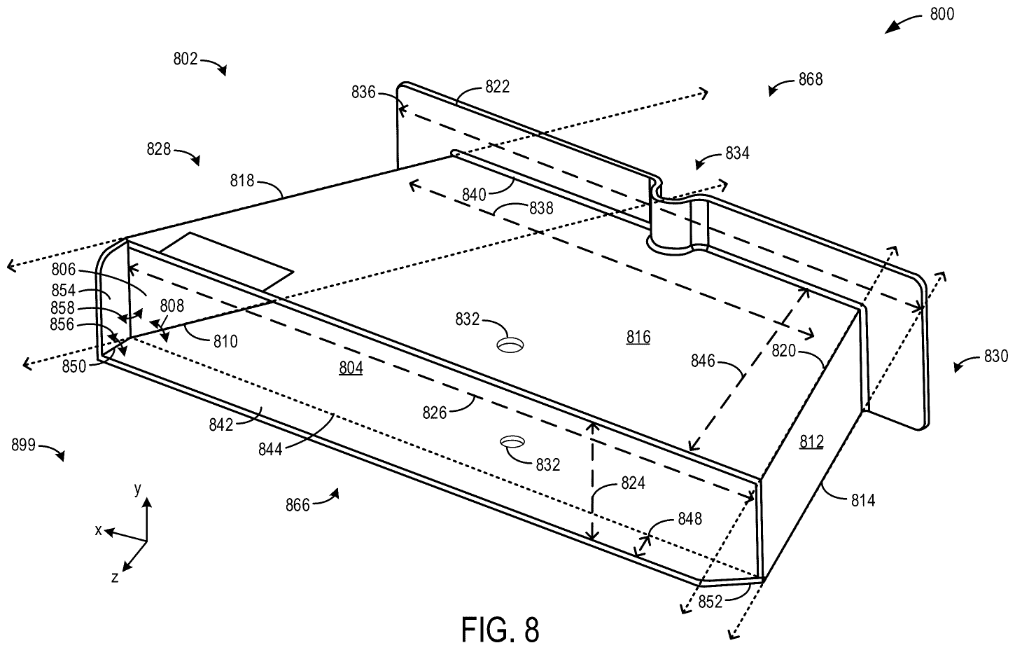

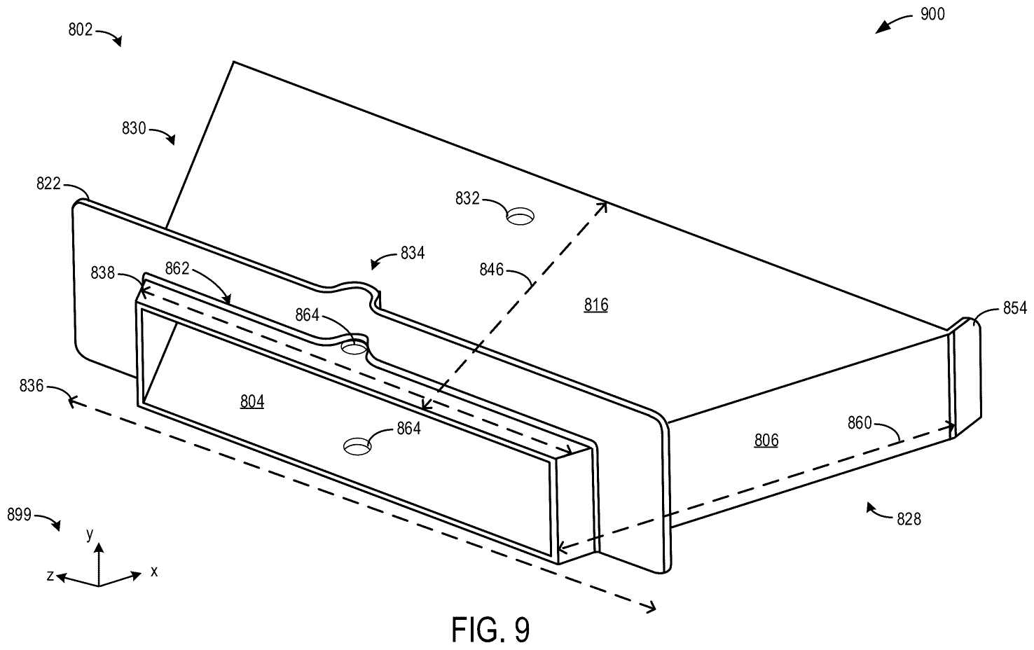

Turning to FIGS. 8-9, a third example of a radiation shielding element 802 is shown. FIG. 8 shows a first perspective view 800 and FIG. 9 shows a second perspective view 900 of the radiation shielding element 802. An axis system 899 is provided in FIGS. 8-9 for reference, and FIGS. 8-9 are described simultaneously herein. The y-axis may be a vertical axis (e.g., parallel to a gravitational axis), the x-axis may be a lateral axis (e.g., horizontal axis), and the z-axis may be a longitudinal axis, in one example. However, the axes may have other orientations, in other examples.

The radiation shielding element 802 is a single continuous shape formed of a first wall 804 and a second wall 806 positioned at a non-zero angle 808 relative to the first wall 804, the second wall 806 continuous with the first wall 804 along a first axis 810. Additionally, the radiation shielding element 802 includes a third wall 812 continuous with the first wall 804 along a second axis 814 of the first wall 804 different from the first axis 810, a fourth wall 816 continuous with the second wall 806 along a third axis 818 (e.g., of the second wall 806, opposite from the first axis 810) and continuous with the third wall 812 along a fourth axis 820 (e.g., of the third wall 812, opposite from the second axis 814). The radiation shielding element 802 further includes a fifth wall 822 continuous with each of the first wall 804, the second wall 806, the third wall 812, and the fourth wall 816. Each of the first wall 804, the second wall 806, the third wall 812 and the fourth wall 816 are planar. The second wall 806 and the third wall 812 are rectangular, and the first wall 804 and the fourth wall 816 are substantially trapezoidal. The fifth wall 822 is rectangular and has a curved extension which extends out of plane (e.g., the x-y plane, with respect to the axis system 899), as further described herein.

The radiation shielding element 802 is formed of the tungsten polymer blend of tungsten and a polymer, wherein tungsten is in an amount of 20% to 60% by volume with respect to the polymer. As described above, the polymer may be one of PA11, PA12, TPU, ABS, or an equivalent plastic. The rigidity of the tungsten polymer blend when in a solid form enables the second wall 806 to be formed at the non-zero angle 808 with respect to the first wall 804. For example, the radiation shielding element 802 has the second wall 806 positioned at a 90-degree angle with respect to the first wall 804. In other embodiments, the non-zero angle 808 may be greater than 0-degrees and less than 90-degrees, or greater than 90-degrees and less than 180-degrees. The composition of the tungsten polymer blend (e.g., the percentage of tungsten by volume with respect to polymer) enables the tungsten polymer blend to cool from a molten state to a solid state rapidly (e.g., within 5 seconds), following the dispensing of the tungsten polymer blend in the molten state from an additive fabrication tool, as further described with respect to FIG. 10. In the solid state, the tungsten polymer blend is rigid enough (e.g., has the tensile strength and compressive strength) to support structures positioned at non-zero angles with respect to each other, such as the first wall 804 and the second wall 806 of the radiation shielding element 802.

As is described above with respect to FIGS. 3, 4, and 8, the rigidity of the radiation shielding element as provided by the tungsten polymer blend in the solid state (e.g., not having heat applied thereto) enables formation of the third wall 812 and the fourth wall 816. The fourth wall 816 is spaced apart from the first wall 804 by a height 824 of the second wall 806 and the third wall 812.-The second wall 806 and the third wall 812 are spaced apart by a first length 826 of the first wall 804 and the fourth wall 816 on a first side 866 of the radiation shielding element 802. The second wall 806 and the third wall 812 are further spaced apart by a second length 838 at a second side 868 of the radiation shielding element 802, opposite from the first side 866. The first length 826 and the second length 838 are shown for illustration purposes, and dimensions of the first wall 804 and the fourth wall 816 may be the same or may be different. For example, the first length 826 for the first wall 804 may be greater than the first length 826 of the fourth wall 816 and, as a result, each of the second wall 806 and the third wall 812 may be positioned at a non-zero angle (e.g., the non-zero angle 808), with respect to the first wall 804, which is less than 90-degrees. The second wall 806 and the third wall 812 extend between a non-parallel pair of sides of the trapezoid (e.g., between a third side 828 of each of the first wall 804 and the fourth wall 816, and between a fourth side 830 of each of the first wall 804 and the fourth wall 816). The third wall 812 is formed at a non-zero angle with respect to the first wall 804 and the fourth wall 816, and the fourth wall 816 is formed parallel to the first wall 804, and at a non-zero angle with respect to the second wall 806. In the example of FIG. 8, each of the non-zero angles between walls are equal to 90-degrees.

Additionally, the rigidity of the radiation shielding element as provided by the tungsten polymer blend in the solid state enables formation of extensions from one or more of the first wall 804, the second wall 806, the third wall 812, the fourth wall 816, and the fifth wall 822. The first wall 804 has a substantially trapezoidal shape which is planar and parallel to the fourth wall 816, and further includes a planar extension 842. A dashed line 844 is included for illustrative purposes to show a region of the planar extension 842 and does not indicate a seam or other characteristic of the radiation shielding element 802. With respect to the axis system 899, the first wall 804 and the fourth wall 816 are planar in the x-z plane and positioned parallel. Both a trapezoidal shape of the first wall 804 and the fourth wall 816 have a first width 846, and a second width 848 of the planar extension 842 further extends a total width of the first wall 804 to be greater than the first width 846 of the fourth wall 816. The planar extension 842 has a first edge 850 which forms an approximate 90-degree angle with a bottom edge (e.g., along the dashed line 844) of the first wall 804, and further has a second edge 852 which forms a second non-zero angle (e.g., less than 180-degrees and greater than 90-degrees) with the bottom edge of the first wall 804. A flap extension 854 is continuous with the planar extension 842 of the first wall 804 at a third non-zero angle 856 (e.g., 90-degrees) and continuous with the second wall 806 at a fourth non-zero angle 858 (e.g., less than 180-degrees and greater than 90-degrees).

As described herein with respect to FIGS. 3-8, the tungsten and polymer blend may have a rigidity in the solid state that enables formation of structurally stable geometries. As shown in FIGS. 8 and 9, the flap extension 854 and the planar extension 842 are coupled at the third non-zero angle 856 and are each further coupled to other elements of the radiation shielding element 802 along one additional axis. The rigidity of the solid state tungsten and polymer blend enables the flap extension 854 and the planar extension 842 to be structurally stable (e.g., not bend/sag out of plane) across the respective geometry of the extension.

The first wall 804 and fourth wall 816 may be spaced apart, as shown in FIGS. 8 and 9, without bowing, sagging, or otherwise deforming out of plane (e.g., the x-z plane, with respect to the axis system 899) without support therebetween from support elements other than the second wall 806 and the third wall 812 due to a structural integrity of the first wall 804 and the second wall 806 provided by a rigidity of the tungsten and polymer blend when in the solid state. Additionally, each of the first wall 804 and the fourth wall 816 include a circular through hole 832. In the example of FIG. 8, the circular through hole 832 of each of the first wall 804 and the fourth wall 816 are aligned along the y-axis. In some examples, a wire or other connector of an imaging device (e.g., the CT system 100) may pass through the circular through holes 832. In further examples, a screw or other coupling device may pass through one or both of the circular through holes 832 to couple the radiation shielding element 802 to an imaging device or other radiation emitting source.

A rectangular shape of the fifth wall 822 is planar with respect to the x-y plane of the axis system 899, and a curved extension 834 of the fifth wall 822 extends out of the x-y plane in a direction of the z-axis. The fifth wall 822, including the curved extension 834, is continuous with each of the first wall 804, the second wall 806, the third wall 812, and the fourth wall 816. As shown in FIG. 9, the first width 846 of the trapezoidal shape of the first wall 804 and the fourth wall 816, and a second length 860 of each of the second wall 806 and the third wall 812 extend through a rectangular opening 862 of the fifth wall 822. The fifth wall 822 further extends a wall length 836, which is longer than the second length 838 of the first wall 804 and the fourth wall 816 along a top edge 840. The curved extension 834 is positioned at an approximate center of the wall length 836, and extends in the direction of the z-axis into the fourth wall 816. The curved extension 834 is continuous with both of the fourth wall 816 and the fifth wall 822, and thus the radiation shielding element 802 has no seams or gaps between the fifth wall 822 and the fourth wall 816. Additionally, the first wall 804 and the fourth wall 816 include a second pair of circular through holes 864 which are vertically aligned (e.g., along the y-axis, with respect to axis system 899) with each other and with a concave side of the curved extension 834. Similar to the circular through holes 864 of the first wall 804 and the fourth wall 816, the curved extension 834 of the fifth wall 822 and the second pair of circular through holes 864 may provide a channel through which to pass one or more wires or other coupling elements of a user input device and/or imaging device. For example, the radiation shielding element 802 may be positioned such that the rectangular shape of the fifth wall 822 is positioned in face-sharing contact with a user input device, and the curved extension 834 provides a gap between the user input device and the radiation shielding element 802. Additionally or alternatively, the curved extension 834 and the second pair of circular through holes 864 may be used to couple the radiation shielding element 802 to an imaging device or other radiation emitting source. For example, a screw or other coupling device may be passed through the curved extension 834 and the second pair of circular through holes 864 to couple the radiation shielding element 802 to an imaging device (e.g., the CT system 100) or other radiation emitting source.

As further described with respect to the method of FIG. 10, the radiation shielding element 802 may be formed via additive fabrication in which pellets, powder, spooled filament, or other solid state blend of tungsten and polymer is heated to become molten, and molten blend is extruded from an additive fabrication tool into a predetermined geometry, the molten blend rapidly (e.g., within 5 seconds of being extruded) cooling to solid state. In some embodiments, the additive fabrication method may include dispensing the tungsten and polymer blend in a molten state along an additive fabrication tool pathway, wherein the additive fabrication tool pathway includes a plurality of planar layers which are vertically stacked. For example, referring to FIG. 8, an additive fabrication tool pathway may begin by forming elements of the first wall 804, the second wall 806, the third wall 812, and the fifth wall 822 which are planar in the x-z plane, with respect to the axis system 899, at a y-coordinate of a printing surface of the additive fabrication tool. The additive fabrication tool pathway may then form subsequent planar layers (e.g., in the x-z plane) of the radiation shielding element 802, progressing up the y-axis. In this way, each planar layer, wall, and extension of the radiation shielding element 802 may be formed as a single, continuous, seamless element.

FIG. 10 illustrates a method 1000 for forming a radiation shielding element using additive fabrication and a blend of tungsten and a polymer, wherein tungsten is in an amount of 20% to 60% by volume with respect to the polymer. The polymer is one of polyamide (PA) 11, PA12, TPU, ABS, or an equivalent plastic. As described herein with respect to FIGS. 3-9, the composition of the tungsten polymer blend (e.g., the amount of tungsten with respect to the amount of polymer and the type of polymer) enable the tungsten polymer blend to be heated to a molten state, in which the tungsten polymer blend can be used by an additive fabrication tool to form a geometry of a radiation shielding element. The tungsten polymer blend may rapidly cool to the solid state, for example, within five seconds of being dispensed from the additive fabrication tool in the molten state. In the solid state, composition of the tungsten polymer blend provides a structural rigidity to the radiation shielding element, enabling formation of complex geometries including a first wall and a second wall positioned at a non-zero angle relative to the first wall, the second wall continuous with the first wall along a first axis, as well as additional third, fourth, and fifth walls, undercuts, grooves, through holes, flaps, extensions, and so on. A density of the tungsten polymer blend which forms the radiation shielding element may be between 4000 kg/m3 and 12000 kg/m3. As described with respect to FIGS. 5-6, a material density of the tungsten polymer blend may be adjusted (e.g., the tungsten polymer blend may have a variable material density within the 4000 kg/m3 to 12000 kg/m3 range) by adjusting a thickness of walls of the radiation shielding element. The thickness of the walls may be provided as a parameter of the radiation shielding element to the additive fabrication tool, as further described herein. In this way, a desired level of shielding may be provided by a radiation shielding element which is formed, via additive fabrication, of the tungsten polymer blend of tungsten and a polymer, wherein tungsten is in an amount of 20% to 60% by volume with respect to the polymer, which further enables the radiation shielding element to have complex geometries used to provide more radiation shielding compared to conventional, planar radiation shielding elements.

The method 1000 described further herein may be stored as executable instructions in non-transitory memory on a computing device (or controller). The computing device may be coupled to one or more of a user input device and an additive fabrication tool via a wired or a wireless connection. The additive fabrication tool may be provided with the tungsten polymer blend of tungsten and polymer in one or more of a powder form, a pellet form, or a filament form (e.g., spooled filament).

At 1002, the method 1000 includes receiving data defining one or more parameters of a radiation shielding element. The data may be received by the device from the user input device, where a user may input the one or more parameters. In other embodiments, the data may be received by the device from a computer or other data storage device coupled to the device via a wired and/or wireless connection. The data defines one or more parameters of the radiation shielding element which can be used to design a model representing a geometry of the radiation shielding element. For example, the parameters can include a number of walls; an angle between each of the walls; a number, size, and positioning of one or more through holes; an angle of a curved flap, through hole, or groove; a wall thickness; a shape and size of each wall; and so on.

At 1004, the method 1000 includes generating a digital model of the radiation shielding element from the one or more parameters, the digital model comprising a first wall and a second wall positioned at a non-zero angle relative to the first wall, the second wall continuous with the first wall along a first axis. For example, the non-zero angle may be 90-degrees, greater than 0-degrees and less than 90-degrees, or greater than 90-degrees and less than 180-degrees. The digital model includes information from the one or more parameters and provides a guide for forming the radiation shielding element using additive fabrication. In some embodiments, the digital model may further include a third wall continuous with the first wall along a second axis of the first wall different from the first axis. In addition to the third wall, in further embodiments, the digital model further comprises a fourth wall continuous with the second wall along a third axis of the second wall different from the first axis, and continuous with the third wall along a fourth axis of the third wall different from the second axis.

At 1006, the method 1000 includes defining an additive fabrication tool pathway for forming the radiation shielding element as a single, continuous shape, according to the digital model. As described herein with respect to FIGS. 3-9, the radiation shielding element includes at least the first wall and the second wall, which are continuous with each other and thus are seamless along the first axis at which the first wall and the second wall meet. The additive fabrication tool pathway may include a starting location, an ending location, and one or more locations to pause dispensing of the tungsten polymer blend and move a nozzle or a printing surface of the additive fabrication tool. For example, the additive fabrication tool pathway may indicate the starting location at a bottom left quadrant of the printing surface, and direct the nozzle to move and dispense the tungsten polymer blend of tungsten and polymer in a serpentine, zig-zag pattern such that each row of dispensed material is in the same plane (e.g., a horizontal plane parallel with the printing surface) to form the first wall. Following completion of the first wall, the additive fabrication tool pathway may direct the nozzle to pause dispensing material and move to a location along the first axis of the first wall and resume dispensing material to form the nozzle to form the second wall. As further described herein, dispensing of molten material (e.g., the tungsten polymer blend of tungsten and polymer) may form the second wall as a continuation of the first wall, such that material of the first wall and the second wall are fused. In some embodiments, the additive fabrication tool may have more than one nozzle and the additive fabrication tool pathway may include a pathway for each nozzle.

At 1008, the method 1000 includes providing a tungsten polymer blend to the additive fabrication tool wherein the tungsten is in an amount of 20% to 60% by volume with respect to the polymer. Providing the tungsten polymer blend to the additive fabrication tool may include commanding the additive fabrication tool, which is communicably coupled to the computing device executing the method 1000, to load the tungsten polymer blend from a storage device (e.g., from a spool of filament, from a reservoir of powder or pellets, and so on) into a dispensing element of the additive fabrication tool (e.g., a nozzle). In some embodiments, the tungsten polymer blend may be provided as a molten compound, where a substantially pure volume of tungsten is substantially evenly distributed throughout a polymeric mixture. In further embodiments, the tungsten polymer blend may be provided to the additive fabrication tool in one or more of a solid state, for example, a powder form, a pellet form, or a filament form (e.g., spooled filament), and the tungsten polymer blend may be heated by the additive fabrication tool to a molten state. For example, the filament (e.g., solid state) may be fed into a nozzle of the additive fabrication tool, and the nozzle may heat the filament to a temperature at which the tungsten polymer blend becomes molten, with the tungsten substantially evenly distributed throughout the polymeric mixture. For example, a temperature of molding (e.g., at which the tungsten polymer blend is in a molten state) is between 200° C. and 300° C., depending upon the application of usage and method of manufacturing the radiation shielding element and processes.

At 1010, the method 1000 includes actuating the additive fabrication tool to dispense the molten compound into a predetermined location according to the additive fabrication tool pathway. As described with respect to operation 1006, the predetermined location defines a single, continuous geometry of the radiation shielding element having the first wall and the second wall positioned at the non-zero angle relative to the first wall, the second wall continuous with the first wall along the first axis. In embodiments where the digital model includes a third wall, dispensing the molten compound from the additive fabrication tool into the predetermined location according to the additive fabrication tool pathway may include dispending the molten compound to form the third wall following completion of dispensing from the additive fabrication tool the molten compound into the predetermined location according to the additive fabrication tool pathway to form the first wall. In embodiments where the digital model includes the third wall and a fourth wall, dispensing the molten compound from the additive fabrication tool into the predetermined location according to the additive fabrication tool pathway may include dispending the molten compound to form the fourth wall following completion of dispensing from the additive fabrication tool the molten compound into the predetermined location according to the additive fabrication tool pathway to form the second wall and the third wall.

The molten compound may be dispensed out of one or more nozzles of the additive fabrication tool onto the printing surface. For example, dispensing the molten compound from the additive fabrication tool into the predetermined location according to the additive fabrication tool pathway may include dispending the molten compound simultaneously from a first nozzle and a second nozzle of the additive fabrication tool to form the first wall and the second wall, respectively. In other embodiments, dispensing the molten compound from the additive fabrication tool into the predetermined location according to the additive fabrication tool pathway comprises dispending the molten compound to form the first wall, then dispensing the molten compound to form the second wall. As described herein, the molten compound may rapidly cool from the molten state to the solid state (e.g., within 5 seconds of being dispensed), thus forming a structurally rigid geometry of the radiation shielding element.

The technical effect of a single, continuous radiation shielding element formed of a blend of tungsten and polymer, wherein an amount of tungsten is 20% to 60% by volume with respect to the polymer is radiation shielding which is comparable to shielding provided by lead or other tungsten and polymer blend material provided by materials which are widely available and easily able to be shaped into a wide variety of complex shapes while still being structurally stable.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A radiation shielding element having a single continuous shape formed of a first wall and a second wall positioned at a non-zero angle relative to the first wall, the second wall continuous with the first wall along a first axis, and both of the first wall and the second wall comprised of a tungsten polymer blend, wherein tungsten is in an amount of 20% to 60% by volume with respect to polymer.

2. The radiation shielding element of claim 1, further comprising at least one of an undercut, a groove, a flap, or a through hole.

3. The radiation shielding element of claim 1, wherein each of the first wall and the second wall are planar.

4. The radiation shielding element of claim 1, wherein the first wall and the second wall of the radiation shielding element are each between 0.2 mm and 6 mm.

5. The radiation shielding element of claim 1, further comprising a third wall continuous with the first wall along a second axis of the first wall different from the first axis, the third wall comprised of the tungsten polymer blend of tungsten and the polymer, wherein tungsten is in the amount of 20% to 60% by volume with respect to the polymer.

6. The radiation shielding element of claim 5, further comprising a fourth wall continuous with the second wall along a third axis of the second wall different from the first axis, and continuous with the third wall along a fourth axis of the third wall different from the second axis, the fourth wall comprised of the tungsten polymer blend of tungsten and the polymer, wherein tungsten is in the amount of 20% to 60% by volume with respect to the polymer.

7. The radiation shielding element of claim 1, wherein the polymer is one of polyamide (PA) 11, PA12, thermoplastic polyurethane (TPU), acrylonitrile butadiene styrene (ABS), or an equivalent plastic.

8. The radiation shielding element of claim 1, wherein the tungsten polymer blend of tungsten and the polymer is able to transition from a solid state to a molten state via exposure to heat.

9. The radiation shielding element of claim 1, wherein the tungsten polymer blend of tungsten and the polymer is in one of a powder form, a pellet form, or a filament form prior to being used to form the radiation shielding element.

10. The radiation shielding element of claim 1, wherein a density of the tungsten polymer blend of tungsten and the polymer is between 4000 kg/m³ and 12000 kg/m³.

11. A method of manufacturing a radiation shielding element, comprising:
receiving data defining one or more parameters of a radiation shielding element;
generating a digital model of the radiation shielding element from the one or more parameters, the digital model comprising a first wall and a second wall positioned at a non-zero angle relative to the first wall, the second wall continuous with the first wall along a first axis;
defining an additive fabrication tool pathway for forming the radiation shielding element as a single, continuous shape according to the digital model;
providing a tungsten polymer blend to an additive fabrication tool, the tungsten polymer blend comprising a substantially pure volume of tungsten substantially evenly distributed throughout a polymeric mixture, wherein the tungsten is in an amount of 20% to 60% by volume with respect to the polymeric mixture; and
actuating the additive fabrication tool to dispense the tungsten polymer blend in a molten state into a predetermined location according to the additive fabrication tool pathway, wherein the predetermined location defines a single, continuous geometry of the radiation shielding element having the first wall and the second wall positioned at the non-zero angle relative to the first wall, the second wall continuous with the first wall along the first axis.

12. The method of claim 11, wherein dispensing the tungsten polymer blend from the additive fabrication tool in the molten state comprises directing the additive fabrication tool to heat the tungsten polymer blend from a solid state to the molten state, and dispense the tungsten polymer blend in the molten state from a nozzle and a second nozzle of the additive fabrication tool.

13. The method of claim 11, wherein providing the tungsten polymer blend to the additive fabrication tool includes providing the tungsten polymer blend in the molten state to the additive fabrication tool.

14. The method of claim 11, wherein the digital model further comprises a third wall continuous with the first wall along a second axis of the first wall different from the first axis, and dispensing from the additive fabrication tool the tungsten polymer blend into the predetermined location according to the additive fabrication tool pathway comprises dispending the tungsten polymer blend to form the third wall following completion of dispensing from the additive fabrication tool the tungsten polymer blend into the predetermined location according to the additive fabrication tool pathway to form the first wall.

15. The method of claim 14, wherein the digital model further comprises a fourth wall continuous with the second wall along a third axis of the second wall different from the first axis, and continuous with the third wall along a fourth axis of the third wall different from the second axis, and dispensing from the additive fabrication tool the tungsten polymer blend into the predetermined location according to the additive fabrication tool pathway comprises dispending the tungsten polymer blend to form the fourth wall following completion of dispensing from the additive fabrication tool the tungsten polymer blend into the predetermined location according to the additive fabrication tool pathway to form the second wall and the third wall.

16. A radiation shielding element, comprising:
a first planar wall; and
a second planar wall continuous with the first planar wall along a first axis and positioned at a non-zero angle relative to the first planar wall, wherein each of the first planar wall and the second planar wall have at least one of an undercut, a groove, a flap, or a through hole, and are comprised of a blend of tungsten and a polymer, wherein tungsten is in an amount of 20% to 60% by volume with respect to the polymer.

17. The radiation shielding element of claim 16, wherein the non-zero angle is 90 degrees.

18. The radiation shielding element of claim 16, wherein the non-zero angle is greater than 0 degrees and less than 90 degrees.

19. The radiation shielding element of claim 16, wherein the non-zero angle is greater than 90 degrees and less than 180 degrees.

20. The radiation shielding element of claim 16, further comprising a third planar wall positioned perpendicular to the first planar wall in a first plane and positioned perpendicular to the second planar wall in a second plane, different from the first plane, the third planar wall continuous with each of the first planar wall and the second planar wall.
